(19) United States
(12) Patent Application Publication
(10) Pub. No.: US 2026/0327535 A1
(43) Pub. Date: Oct. 22, 2026

(54) CARBON REMOVAL OR CARBON CAPTURE USING MICROALGAE CULTIVATED IN SCALABLE PHOTOBIOREACTORS PAIRED WITH A PARTICLE FILTRATION SYSTEM

(71) Applicants: Antonin AFFHOLDER, Tucson, AZ (US); Daniel APAI, Tucson, AZ (US); Joel L. CUELLO, Tucson, AZ (US); Arizona Board of Regents on Behalf of the University of Arizona, Tucson, AZ (US)

(72) Inventors: Antonin Affholder, Tucson, AZ (US); Daniel Apai, Tucson, AZ (US); Joel L. Cuello, Tucson, AZ (US)

(73) Assignee: Arizona Board of Regents on Behalf of the University of Arizona, Tucson, AZ (US)

(21) Appl. No.: 19/486,145

(22) PCT Filed: May 24, 2024
(86) PCT No.: PCT/US2024/031078

§ 371 Date: Nov. 20, 2025

Related U.S. Application Data
(60) Provisional application No. 63/468,593, filed on May 24, 2023.

(51) Int. Cl. C12M 1/00; C12M 1/26; C12N 1/125; C12R 1/89

(52) U.S. Cl. CPC C12M 21/02; C12M 23/58; C12M 33/14; C12M 47/10; C12N 1/125; C12R 2001/89

(57) ABSTRACT

A system including a combination of a photobioreactor fluidly coupled to a particle filtration system is provided. Also provided are methods of cultivating Coccolithophores in the system, which can be used to capture carbon dioxide in the culture medium into calcium carbonate.

### CROSS REFERENCE TO RELATED APPLICATIONS

This application claims priority to U.S. Provisional Application No. 63/468,593, filed May 24, 2023, which is incorporated by reference in its entirety.

### FIELD

This disclosure relates to photobioreactor systems, particularly photobioreactors coupled to a particle filtration system, and methods of their use, for example for cell culture.

### BACKGROUND

Through its absorption of thermal infrared emission, atmospheric CO₂ traps heat and significantly contributes to global climate change. Although there is global agreement that the rise of atmospheric CO₂ levels must be curtailed to avoid catastrophic levels of warming within the current century, the needed reduction in CO₂ emission rates is significantly lagging. Recent assessments by the United Nations Intergovernmental Panel on Climate Change (IPCC) suggests that, in a broad range of future climate change projections, a large-scale (CO₂-Gigaton-level, CGt) active capture and removal of atmospheric carbon dioxide will be necessary.

A multitude of active, small-scale atmospheric carbon dioxide extraction systems currently exist. Fundamentally, there are three challenges to scaling up these systems to CGt-levels. First, most of these systems are based on chemical filtration, with energy supplied from renewable sources. Such chemical filtration-based approaches led to functional, effective, and demonstrated systems. However, such systems are intrinsically energy-intensive and require major up-front investment for installation and continuous mechanical and chemical maintenance. Unit costs for such systems remain high regardless of how many are built; thus, they cannot be cost-efficiently scaled up to large volumes. Second, the storage of captured or extracted CO₂ poses a significant problem. Unless CO₂ is chemically transformed (dissociated or bound to other molecules), it remains difficult and expensive to store in large volumes safely and over long periods of time (such as decades/centuries). Meanwhile, chemically transforming CO₂ requires significant amounts of energy (which currently comes with its own net CO₂ emission) and, thus, costly. Third, chemical solutions pose environmental and economic risks when scaled up. For example, the increased demand for large volumes of chemical catalysts may drive up costs worldwide and their large-scale extraction (through mining operations) may have significant environmental impact. Similarly, the unavoidable by-products of large-scale chemical operations may lead to difficult-to-control environmental impact. Thus, operational safety for such large-scale chemical extraction facilities will likely be among its cost drivers.

Given these three fundamental challenges, it is not foreseen that existing approaches could ever reach the CGt/year-level extraction volumes that are required to contribute to the mitigation of climate change. Thus, there is a global and yet unmet need for a carbon dioxide extraction method that is: (1) Intrinsically scalable, e.g., unit costs (including fabrication, installation, and operation) significantly decrease with increasing number of units; (2) Transforms the extracted CO₂ into a stable, non-gaseous chemical compound that can be safely and cost-effective stored (or, even, re-used); and (3) Relies on or approximates naturally occurring processes to minimize its environmental impact.

### SUMMARY

Provided herein is a scalable solution to efficiently extract CO₂ from ocean water (a gigantic reservoir of CO₂ that is coupled to the atmosphere), the atmosphere, or point sources (e.g., points of emission). The disclosed carbon capture or carbon removal system uses a combination of photobioreactor and particle filtration system, in combination with calcium carbonate shell-forming photosynthetic algae, (e. g., Coccolithophores), to transform dissolved CO₂ from the ocean or other sources into calcium carbonate (mineral) blocks. These mineral blocks can be easily transported, safely stored or, after further processing, utilized for commercial purposes, such as building blocks. The primary energy demands of the system are met by solar irradiation, as the capture and chemical processing is carried out by photosynthetic algae. The photosynthetic, shell-forming Coccolithophores can, under suitable or optimized conditions, reproduce quickly, doubling in number several times a day. Therefore, the system represents a minimal environmental impact for its scope and offers a solution for carbon dioxide extraction with a strong potential to be cost-effectively scalable.

The disclosed system includes a photobioreactor coupled to a particle filtration system. In some examples, the photobioreactor is an Accordion photobioreactor, an Air Accordion photobioreactor, an Air Loop Accordion photobioreactor, or an axial dispersion photobioreactor. In some examples, the photobioreactor is directly connected to the filtration system. In other examples, the photobioreactor and filtration system are not connected, but are used sequentially.

Also provided are methods of cultivating carbon-fixing algae (such as Coccolithophores) in the disclosed systems. In some examples, the methods include culturing Coccolithophores in the photobioreactor under conditions suitable for their growth and/or formation of coccoliths, for example in a suitable culture medium. The methods also include filtering the culture medium (and included Coccolithophores) through the particle filtration system.

The foregoing and other features of the disclosure will become more apparent from the following detailed description, which proceeds with reference to the accompanying figures.

### BRIEF DESCRIPTION OF THE DRAWINGS

FIG. 1 is a diagram illustrating Coccolithophore carbon chemistry. When atmospheric CO₂ enters the sea surface (1), it undergoes a series of reversible chemical reactions known as the seawater carbonate buffer system (2), which releases protons (H⁺) that acidify the sea water. Coccolithophores and other algae assimilate CO₂ to produce organic carbon through photosynthesis (3). Coccolithophores also perform calcification reactions, in which two bicarbonate ions (HCO₃⁻) are converted into one calcium carbonate (CaCO₃) and one CO₂ molecule (4). The CaCO₃ is incorporated into coccoliths in the algal shell. The CO₂ from calcification is released, and can either contribute to ocean acidification or degas back to the atmosphere (5). Adapted from Hutchins, Nature 476:41-42, 2011.

FIG. 2 is a diagram of an exemplary system illustrating the aspect of use of gas manifolds to deliver CO₂-containing air into the photobioreactors through use of air compressors and pumps directly from air or from point/concentrated CO₂ sources.

FIG. 3 is a schematic diagram of an exemplary system, illustrating the inclusion of a particle filtration system paired with the photobioreactor to provide a continuous, low-cost filtration capability.

FIG. 4 is a graph showing alkalinity and calcium yield obtained using different calcium sources from several categories—industry (including lime, cement, cement kiln dust, slag blast furnace, slag steel, biomass ash, lignite ash); natural rocks (including phoscorite and melilitite), and miscellaneous sources (including pure calcium oxide, hydroxyapatite, and CaSiO₃).

FIG. 5 is a plot showing the effect of different combinations of inputs on alkalinity and calcium availability.

### DETAILED DESCRIPTION

The disclosed systems provide several advantages over previous methods of growing Coccolithophores or other photosynthetic or carbon fixing microorganisms (such as microalgae or cyanobacteria) for carbon capture or atmospheric carbon dioxide removal. Use of photobioreactors as described herein allow for a greater level of control over the growth environment than open-air aquaculture systems and, thus, can sustain orders-of-magnitude higher cell density and yields. In addition, extraction of coccolithophore shells from open-air aquacultures is a challenging and costly step; whereas in the disclosed systems, the filtration system can be integrated into the bioreactors and offers a continuous, low-cost extraction mechanism.

The bioreactor systems disclosed herein provide highly efficient biomass production in a closed or substantially closed system with advantages of reduced costs for construction and operation (for example, low cost materials and reduced energy and water requirements), simplicity, modularity, and flexibility. Although bioreactor systems and methods are described herein primarily with respect to Coccolithophore culture, the disclosed systems and methods in their several aspects are also suitable for culture of other photosynthetic and/or carbon fixing cells.

### I. General Considerations

For purposes of this description, certain aspects, advantages, and features of this disclosure are described herein. The disclosed methods, apparatus, and systems should not be construed as being limiting in any way. Instead, the present disclosure is directed toward all novel and nonobvious features and aspects of the various disclosed aspects or examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed aspects are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the term “coupled” generally means physically, mechanically, chemically, magnetically, and/or electrically coupled or linked and does not exclude the presence of intermediate elements between the coupled or associated items absent specific contrary language.

In some examples, values, procedures, or systems may be referred to as “lowest,” “best,” “minimum,” or the like. It will be appreciated that such descriptions are intended to indicate that a selection among many alternatives can be made, and such selections need not be better, smaller, or otherwise preferable to other selections.

In the description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “comprises” means “includes.” In case of conflict, the present specification, including explanations of terms, will control. In addition, the materials, methods, and examples are illustrative only and not intended to be limiting.

### II. Carbon Capture Systems

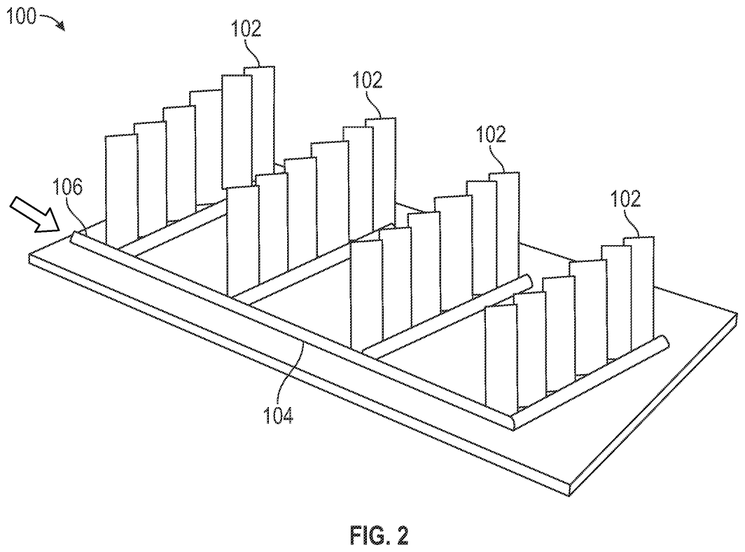

The disclosed system includes a photobioreactor coupled to a particle sedimentation and filtration system. FIG. 2 illustrates an exemplary system 100, including multiple photobioreactors 102 arrayed in a parallel configuration. The system also includes a means for introducing a gas (such as a gas manifold) to each of the photobioreactors 104 and a means (such as a valve or inlet) 106 for introducing gas (such as CO₂) to the system.

In some examples, the system may include one or more photobioreactors (such as 1, 2, 3, 4, 5, or more) that are arrayed in parallel, or in other spatial configurations for increasing or optimizing Coccolithophore growth and/or coccolith production. In some non-limiting examples, the photobioreactor(s) include a tube-like structure aligned on a vertical axis, wherein at least a portion of the container is oriented at an angle relative to the vertical axis, wherein the angle is from about 0° to 90°. In some examples, the container is placed on a support structure with portions oriented at alternating angles, such that the configuration suggests an accordion (e.g., FIG. 2). The photobioreactor also includes a means for introducing a gas including carbon dioxide and/or providing mixing connected to the system.

A photobioreactor is a closed (or substantially closed) system that can be used for cultivating photosynthetic organisms. In some examples, the photobioreactor is an Accordion photobioreactor (e.g., U.S. Pat. No. 8,709,808; incorporated herein by reference in its entirety) an Air Accordion photobioreactor (e.g., U.S. Pat. No. 10,829,725; incorporated herein by reference in its entirety), an Air Loop Accordion photobioreactor (e.g., U.S. Pat. No. 10,604,732, incorporated herein by reference in its entirety), or an axial dispersion photobioreactor (e.g., U.S. Pat. Publ. No. 2020/0318052, incorporated herein by reference in its entirety). In some examples, the photobioreactor is an Air Accordion photobioreactor. Advantages of using Accordion photobioreactor or Air Accordion photobioreactor include (1) Lower material cost compared with conventional photobioreactors, as these have simple frame structure and utilize low-cost low density polyethylene vessel material; (2) Lower power demand, significantly lower pressure drop compared with tubular reactors, and superior liquid mixing and gas mass transfer; (3) Ease of scale up due to modularity in configuration of reactor vessel, flexibility in height, width, and depth of reactor vessels, and use of commercially available parts which can be easily replaced; (4) High resilience against contamination (no sterilization required for food-grade low-density polyethylene); and (5) Low Shear Forces, allowing cultivation of cells that are sensitive to shear forces.

The disclosed systems include at least one opening that allows for the introduction of gases, nutrients, and/or other inputs. The system may also include one or more additional openings or ports for air venting, adding or removing cell culture medium and/or cells, or sensor placement. In some examples, gas (including carbon dioxide), nutrients, and/or other components are introduced in such a way as to provide mixing and aeration of a cell culture in the bioreactor. The air introduction mechanism may be placed any location in the system, including at the top or the bottom of the photobioreactor. An exemplary gas introduction system is illustrated in FIG. 2. In some examples, the gas introduction system may include an air/gas compressor and a gas sparger. In one example, the gas sparger is located at the bottom of the photobioreactor.

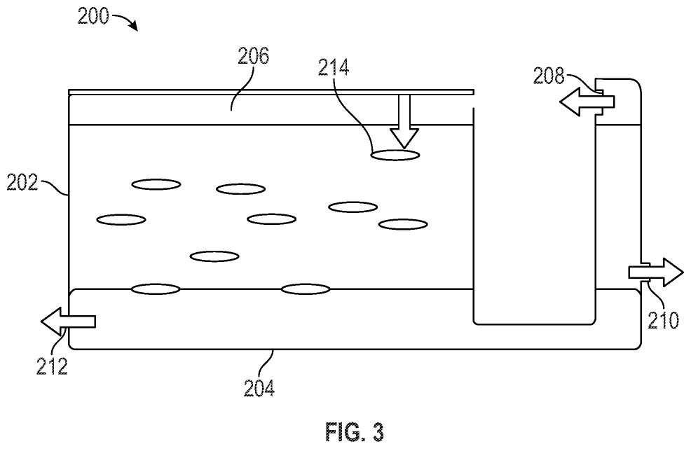

The disclosed systems also include a particle filtration system paired with (e.g., fluidly coupled to) the photobioreactor to provide continuous, low-cost filtration capability. FIG. 3 illustrates an exemplary system including a filtration system 200. The system includes at least one photobioreactor 202, a filtration system 204 fluidly coupled to the photobioreactor 202, a means for introducing a gas to the photobioreactor 206 and a means (such as a valve) for introducing the gas to the system 208. The system also includes one or more ports or outlets, such as an outlet 210 for removing liquid outputs, such as filtered water or culture medium and an outlet 212 for removing solid outputs, such as CaCO₃ or filtration materials. In some examples, the system includes coccolithospores 214, for example in a liquid medium.

The particles to be filtered (e.g., single coccoliths and coccolith clusters) range in diameter from about 1 μm to about 20-40 μm. In some examples, the particle filtration system is fluidly coupled to the bottom of the photobioreactor. In some examples, the filtration system is connected to an output port of the photobioreactor and filters out coccolithophore shells after an initial sedimentation process. The particle filtration system can be any suitable cell filtration or separation system, including a system that utilizes centrifugation or sonication, as well as a bed filtration system.

In some examples, the system utilizes a fine sand filtration system, where water pressure moves fluid from the photobioreactor through a chamber that incorporates a layer of inert particles that form a barrier and filter out particles from the water flow. In some examples, a sand layer is used as the filter, but any low-cost, approximately uniform-size-distribution, inert particles can provide equivalent functionality. Coccoliths filtered out are incorporated into the particle barrier (‘filter’), initially further increasing its filtration capability.

In some aspects one or more sensors are optionally included in the system, for example, in or connected to the photobioreactor. In some examples, sensors and instrumentation are included to monitor one or more parameters of the cell culture, the environment, or both. Culture parameters that may be monitored include water (or other liquid) temperature, electrical conductivity, pH, carbon dioxide, dissolved oxygen, optical density (e.g., cell density), ion concentration (e.g., calcium concentration), and flow rate. Environmental parameters that may be monitored include air temperature, relative humidity, solar radiation, photosynthetic active radiation, and wind speed. One of skill in the art can identify additional parameters that may be desirable to monitor, depending on the size, type, or location of the bioreactor or the type of cell culture being carried out. The sensors for measuring culture parameters are placed in one or more locations in or connected to the system. One or more sensors for monitoring environmental parameters may also be placed in close proximity to the bioreactor, such as within at least 50 meters of the bioreactor.

### III. Methods of Use

The disclosed systems can be utilized to culture organisms (such as Coccolithophores) that can capture carbon dioxide and convert it to calcium carbonate.

A coccolithophore (or coccolithophorid) is a unicellular, eukaryotic phytoplankton or alga, belonging either to the kingdom Protista or Hacrobia, depending on the classification system. Coccolithophores are distinguished by their distinctive calcium carbonate plates (or scales) of uncertain function called coccoliths, which are also important microfossils. Coccolithophores are almost exclusively marine, are photosynthetic, and exist in large numbers throughout the sunlight (photic) zone of the ocean. The most abundant species of coccolithophore, Emiliania huxleyi, is found in temperate, subtropical and tropical oceans, making it an important part of the planktonic base of a large proportion of marine food webs. It is also the fastest growing coccolithophore in laboratory cultures.

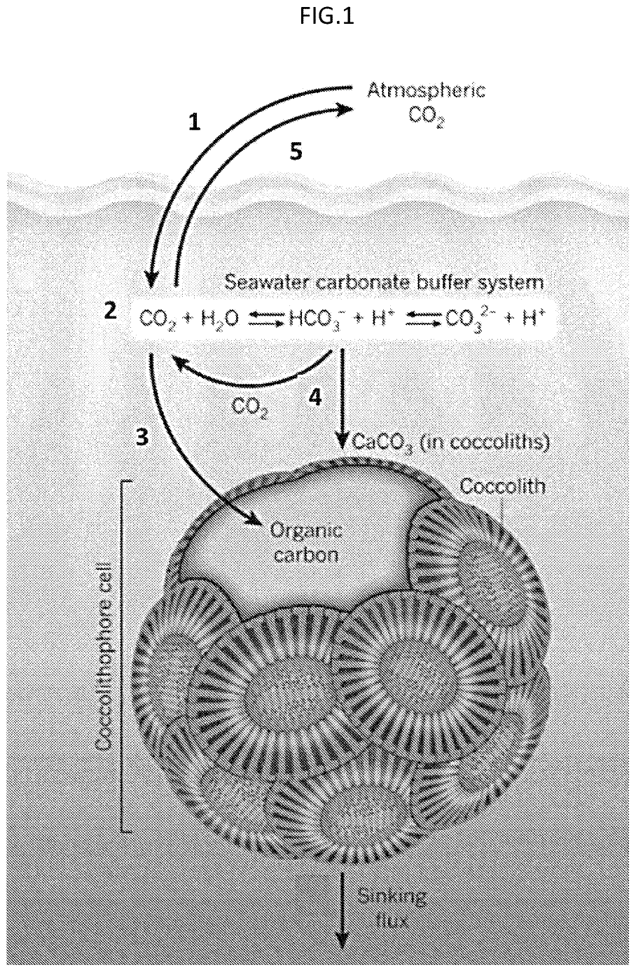

Coccolithophores make their coccoliths out of one part carbon, one part calcium and three parts oxygen (CaCO₃). Thus, each time a molecule of coccolith is made, one less carbon atom is allowed to roam freely in the atmosphere to form greenhouse gases and contribute to global warming (FIG. 1). For every ton of coccoliths produced, 320 pounds of carbon is incorporated or sequestered into the coccoliths (NASA Earth Observatory 1999). Geological and fossil evidence demonstrates that the sedimentation coccoliths can lead to massive CaCO₃ deposits which can remain stable for tens to hundreds of millions of years. For example, the white cliffs of Dover are a well-known coastline that formed through the accumulation of about five hundred meter-high strata of coccoliths, now exposed at a coastline over 8 km in length.

In some aspects, one or more species of Coccolithophores (such as 1, 2, 3, 4, or more) are cultivated in a disclosed system. In some examples, the Coccolithophores include Emiliana huxleyi; however other coccolithophore species can be used as well. Other exemplary species that can be used include Gephyrosapsa oceanica, Calcidiscus leptoporus, Umbilicosphaera foliosa, Oolithotus fragilis, Coccolithus pelagicus, Cruciplacolithus neohelis, and Pleurochrysis carterae. In other examples, one or more species of photosynthetic or carbon fixing microorganisms, such as one or more additional microalgae or cyanobacteria species are cultivated in a disclosed system. Combinations of additional microalgae, Coccolithophores, and cyanobacteria species can also be used (such as 2, 3, 4, or more species).

The Coccolithophores are grown in the photobioreactor under conditions suitable for their growth and/or formation of coccoliths, for example in a suitable culture medium. The conditions suitable for the growth and/or formation of the coccoliths include levels of light intensity (photosynthetic photon flux), light daily duration (photoperiod); light quality (various wavelengths, e.g., proportions of red, blue, white, etc.), aeration volumetric flow rate, CO₂ concentration (e.g., in input gas), ambient temperature, culture medium nutrient composition (including calcium), and culture nutrient medium pH, and can be adjusted as appropriate.

In some aspects, the conditions suitable for growth and/or formation of coccoliths include providing calcium and carbon dioxide to the culture medium of the Coccolithophores in the photobioreactor. Sourcing or partial sourcing of calcium (and other nutrients) required for the growth of the Coccolithophores and/or formation of coccoliths may be from the by-product brine or saline wastewater of desalination systems. In additional examples, the calcium source may include one or more (such as 1, 2, 3, or more) of hydroxyapatite, lignite ash, biomass ash, phoscorite, melilitite, steel slag, blast furnace slag, cement kiln dust, CaSiO₃, cement, lime, or calcium oxide. In some specific examples, the calcium source is cement kiln dust, lignite ash, biomass ash, or cement. In some non-limiting examples, the calcium source may include biomass ash or slag products and hydroxyapatite (e.g., about 20% hydroxyapatite). In other non-limiting examples, the calcium source may include biomass ash or slag products and calcium sulfate (e.g., about 20% calcium sulfate or gypsum). These combinations can improve the yield per kg, for example, by about 5-30% (such as about 5%, about 10%, about 15%, about 20%, about 25%, about 30%, or more) compared to a single calcium source.

In some aspects, the calcium-containing material is introduced into the system directly, for example, after one or more optional pre-processing steps. In some examples, pre-processing includes breaking up larger blocks of materials into smaller blocks or pulverizing via mechanical processing. In other examples, pre-processing includes chemical processing to break down the material's strength, increase its porosity to enable microalgae to access the calcium, or other mechanical, electrical, chemical fragmentation processes. In further examples, pre-processing includes chemical balancing and/or neutralization of potential toxins or chemicals that could compromise the efficiency or viability of microalgae. After optional pre-processing, the material is introduced and/or dissolved into the water which is mixed and injected into the photobioreactor.

In other aspects the input streams may include one or more of filtered and/or recycled water, aerated water (CO₂ injection), nutrient streams (e.g., nitrates, and carriers of phosphorous, iron, calcium). Output streams from the photobioreactor system may include one or more of water or medium (which may be recycled to increase efficiency) and filtered calcium carbonate (which may also include particles from the filtration system).

In some examples, the Coccolithophores are cultured in the photobioreactor under photoautotrophic conditions (by photosynthesis only) or under mixotrophic conditions (combined photosynthesis and heterotrophic production, implemented through addition of exogenous small carbon molecules, e.g., glucose). The photobioreactor may be configured in an open field (e.g., as illustrated in FIG. 2) or may be in a closed or semi-closed environment, such as in a partial or fully covered structure (e.g., in a greenhouse), using materials such as shade cloth, polyethylene, polycarbonate, glass, or other suitable materials. The light source for culturing the Coccolithophores in the photobioreactor may be solar radiation, lamps (such as light emitting diodes), or a combination thereof. The water used in the culture medium may be seawater or other sources.

The methods include introducing carbon dioxide into the photobioreactor during culture of the Coccolithophores. In some examples, the carbon dioxide may be obtained from a point/concentrated source such as an electric power plant, a wastewater treatment plant, or a cement manufacturing plant. In other examples, the carbon dioxide may be from an atmospheric airflow. In one example, the CO₂ is introduced into the system through the mixing of an atmospheric airflow in the water or culture medium contained in the photobioreactor. In other aspects, the water or culture medium entering the photobioreactor is aerated prior to entering the photobioreactor to reduce mechanical stresses to the Coccolithophores. Atmospheric airflow contains in excess of 450 ppm CO₂ and aeration provides efficient equilibration between the water component and the atmospheric CO₂. Depending on the source, the CO₂ concentration in the injected airflow may range from 0.04% to 100%, but its concentration in the growth medium is adjusted to ensure optimal growth conditions. The carbon dioxide is introduced into the culture medium in the photobioreactor using equipment including one or more air compressors, pumps, spargers, and/or gas manifolds.

In some aspects, an air/gas compressor is used to draw in and compress ambient air (containing CO₂), after which the pressurized CO₂-containing air is conveyed into a gas sparger, located at the bottom of the photobioreactor, to be bubbled into the bulk liquid medium inside the photobioreactor in which the Coccolithophore cells are suspended. The volumetric flow rate of the pressurized CO₂-containing air into the photobioreactor is regulated taking into account suitable conditions for cell growth and CO₂ fixation.

In some examples, the conditions for Coccolithophore growth, productivity, and coccolith formation is improved or optimized by applying analysis of data collected from sensors and data collection components included in the system. In some examples, artificial intelligence algorithms are used to analyze the data and determine improved or optimize culture conditions.

The methods further include filtration of the culture medium (including Coccolithophores) through a filtration system. In some examples, water pressure moves the culture medium through the filtration system (such as a sand layer). The water pressure may be provided by a gravitational potential energy (e.g., a passive system) or through the use of a pump. The Coccolithophore shells are retained in the filtration system. Once the coccolithophore shells are filtered, they can be stored or re-used either in combination with the filtering medium or separated from it in an additional process. Such a separation process may be based on sedimentation if the density of the filtering medium is different from that of the coccolithophore shell clusters. The filtered medium can thus be recovered and re-used in the system.

### IV. Overview of Several Aspects

Aspect 1. A carbon capture and/or carbon removal system comprising:
one or more photobioreactors, where the one or more photobioreactors comprise a means for introducing a gas containing carbon dioxide; and
a particle filtration system that is fluidly coupled to the photobioreactor.

Aspect 2. A carbon capture and/or carbon removal system comprising:
one or more photobioreactors, where the one or more photobioreactors comprise a means for introducing a gas containing carbon dioxide; and
a particle filtration system.

Aspect 3. The system of aspect 1 or aspect 2, wherein the photobioreactor comprises a flexible container aligned on a vertical axis, wherein at least a portion of the container is oriented at an angle relative to the vertical axis, wherein the angle is from about 0° to 90°.

Aspect 4. The system of aspect 3, wherein the container is placed on a support structure with portions oriented at alternating angles.

Aspect 5. The system of any one of aspects 1, 3, or 4, wherein the particle filtration system is fluidly coupled to the bottom of the photobioreactor or fluidly coupled to an outlet of the photobioreactor.

Aspect 6. The system of any one of aspects 1 to 5, wherein the particle filtration system comprises sand.

Aspect 7. The system of any one of aspects 1 to 6, wherein the means for introducing the gas comprising carbon dioxide comprises one or more of a gas compressor, gas sparger, and a gas manifold.

Aspect 8. A method of cultivating carbon fixing coccolithophores, comprising culturing one or more coccolithophores in the photobioreactor of the system of any one of aspects 1 to 7 under conditions suitable for growth of the coccolithophores and/or formation of coccoliths.

Aspect 9. The method of aspect 8, wherein the coccolithophores are Emiliania huxleyi.

Aspect 10. The method of aspect 8 or aspect 9, wherein conditions suitable for growth of the coccolithophores and/or formation of coccoliths comprise introducing carbon dioxide into the photobioreactor by the means for introducing the gas comprising the carbon dioxide.

Aspect 11. The method of aspect 10, wherein gas comprising 0.04% to 100% carbon dioxide is introduced into the photobioreactor.

Aspect 12. The method of any one of aspects 8 to 10, wherein the conditions suitable for growth of the coccolithophores and/or formation of coccoliths comprise introducing one or more calcium sources into the photobioreactor.

Aspect 13. The method of aspect 12, wherein the one or more calcium sources comprise lignite ash, biomass ash, cement kiln dust, cement, hydroxyapatite, or calcium sulfate.

Aspect 14. The method of any one of aspects 8 to 13, wherein the photobioreactor is exposed to a light source.

Aspect 15. The method of aspect 14, wherein the light source is sunlight, a light emitting diode, or a combination thereof.

Aspect 16. The method of any one of aspects 8 to 15, further comprising filtering the culture of coccolithophores through the particle filtration system.

Aspect 17. The method of aspect 16, further comprising collecting coccolithophore shells from the particle filtration system and/or recirculating filtered culture medium through the photobioreactor.

### EXAMPLES

The following examples are provided to illustrate particular features of certain aspects of the disclosure, but the scope of the claims should not be limited to those features exemplified.

### Example 1

### Effect of Different Calcium Sources

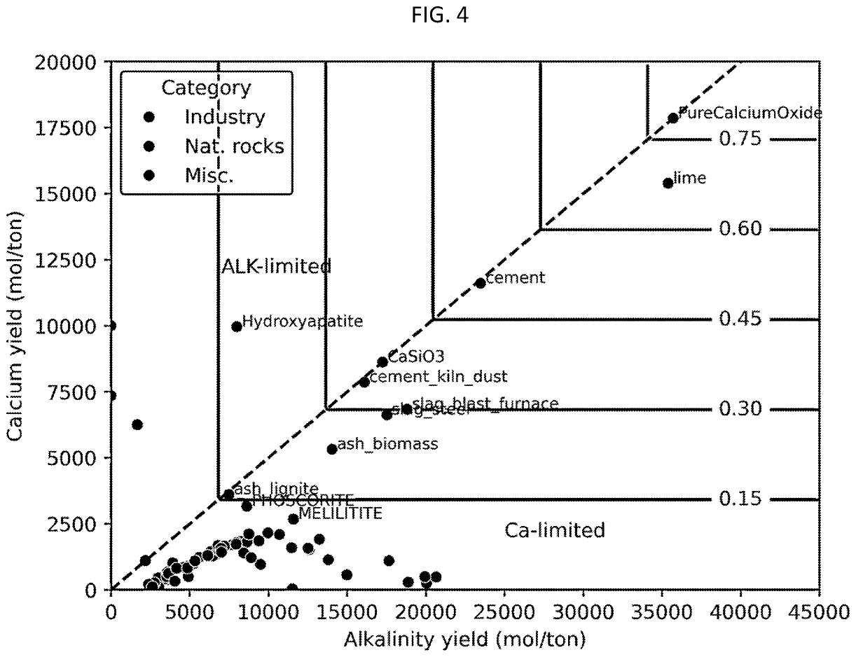

FIG. 4 shows alkalinity yield vs. calcium yield for the indicated materials. For each considered material, published average oxides compositional data (Renforth, Nature Commun. 10:1401, 2019) was used in order to calculate the molar content of calcium and of equivalent alkalinity using the modified Steinour formula (Gunning et al., Waste Management 30:1081-1090, 2010):

f_{ALK}=η(m_{CaO}/M_{CaO}+m_{MgO}/M_{MgO}+m_{Na2O}/M_{NA2O}+m_{K2O}/M_{K2O}−m_{SO3}/M_{SO3}−2 m_{P2O5}/M_{P2O5}−m_{CO2}/M_{CO2})

where m_{X} is the mass fraction of oxide X, M_{X} its molar mass such that fALK is given in moles of alkalinity equivalent per g of a material composed of various oxides. In the calculation η=2 was fixed in order to calculate a maximum alkalinity yield.

The contour lines corresponding to the maximum calcite yield were calculated by taking the minimum of (Ca, ALK/2), where Ca and ALK are molar yields in mol/kg, according to the schematic reaction of calcification:

Ca²⁺+Co₂+H₂O+2ALK⁻>CaCO3

Based on these data, kiln dust, ash (from coal or biomass burning for electricity production), and cement products found in construction/demolition waste are carbon-intensive industrial waste that are not currently fully recycled. FIG. 4 shows that these materials contain abundant calcium and other alkaline materials such that their potential to fuel atmospheric CO₂ removal by calcification in a controlled environment appears promising.

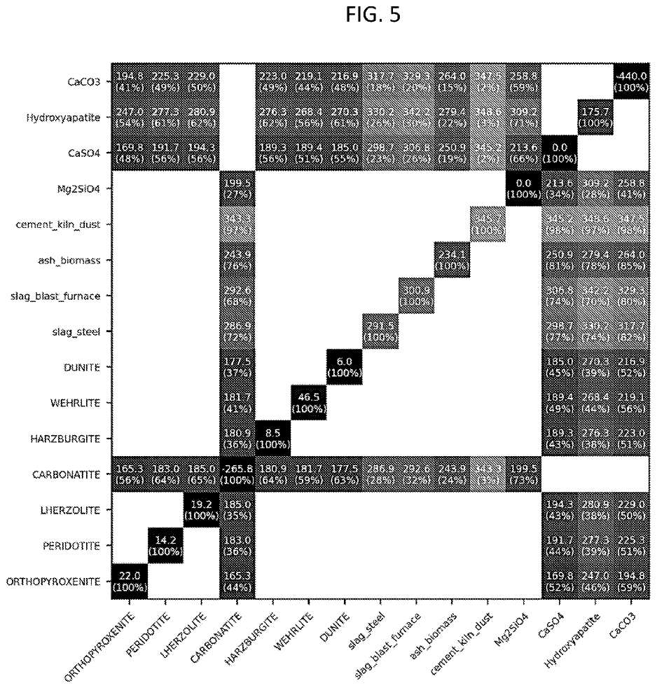

FIG. 5 shows results of a source mixing matrix. Each cell shows the maximum calcite yield (equation above) of a specific mix of a pair of materials corresponding to the column and row of the cell. This specific mix was found by solving:

xALK₁+(1−x) ALK₂=2(xCa₁+(1−x)Ca₂)

where x is the fraction of material 1 (row) in the mix of material 1+material 2 (column). In each cell in the figure, the calcite yield, measured in kgCO₂ is given as an annotation, and the value of x expressed in percent is given in parentheses. Blank cells are pairs of materials for which no solution was found for x in ]0,1[. FIG. 5 shows that complementing some materials with limited amounts of pure minerals can significantly improve their yield in terms of maximum calcite formation. For instance, supplementing biomass ash or slag products with ~20% hydroxyapatite (the main mineral in bones), or calcium sulfate (the main mineral of gypsum) improves the yield per kg of mix by 10% (blast furnace slag+calcite) to 20% (ash+hydroxyapatite).

It will be apparent that the precise details of the systems or methods described may be varied or modified without departing from the spirit of the described aspects of the disclosure. We claim all such modifications and variations that fall within the scope and spirit of the claims below.

1. A carbon capture and/or carbon removal system comprising:
one or more photobioreactors, where the one or more photobioreactors comprise a means for introducing a gas containing carbon dioxide; and
a particle filtration system that is fluidly coupled to the photobioreactor.

2. A carbon capture and/or carbon removal system comprising:
one or more photobioreactors, where the one or more photobioreactors comprise a means for introducing a gas containing carbon dioxide; and
a particle filtration system.

3. The system of claim 1, wherein the photobioreactor comprises a flexible container aligned on a vertical axis, wherein at least a portion of the container is oriented at an angle relative to the vertical axis, wherein the angle is from about 0° to 90°.

4. The system of claim 3, wherein the container is placed on a support structure with portions oriented at alternating angles.

5. The system of claim 1, wherein the particle filtration system is fluidly coupled to the bottom of the photobioreactor or fluidly coupled to an outlet of the photobioreactor.

6. The system of claim 1, wherein the particle filtration system comprises sand.

7. The system of claim 1, wherein the means for introducing the gas comprising carbon dioxide comprises one or more of a gas compressor, gas sparger, and a gas manifold.

8. A method of cultivating carbon fixing coccolithophores, comprising culturing one or more coccolithophores in the photobioreactor of the system of claim 1 under conditions suitable for growth of the coccolithophores and/or formation of coccoliths.

9. The method of claim 8, wherein the coccolithophores are Emiliania huxleyi.

10. The method of claim 8, wherein conditions suitable for growth of the coccolithophores and/or formation of coccoliths comprise introducing carbon dioxide into the photobioreactor by the means for introducing the gas comprising the carbon dioxide.

11. The method of claim 10, wherein gas comprising 0.04% to 100% carbon dioxide is introduced into the photobioreactor.

12. The method of claim 8, wherein the conditions suitable for growth of the coccolithophores and/or formation of coccoliths comprise introducing one or more calcium sources into the photobioreactor.

13. The method of claim 12, wherein the one or more calcium sources comprise lignite ash, biomass ash, cement kiln dust, cement, hydroxyapatite, or calcium sulfate.

14. The method of claim 8, wherein the photobioreactor is exposed to a light source.

15. The method of claim 14, wherein the light source is sunlight, a light emitting diode, or a combination thereof.

16. The method of claim 8, further comprising filtering the culture of coccolithophores through the particle filtration system.

17. The method of claim 16, further comprising collecting coccolithophore shells from the particle filtration system and/or recirculating filtered culture medium through the photobioreactor.
